(19) United States
(12) Patent Application Publication
(10) Pub. No.: US 2025/0082546 A1
(43) Pub. Date: Mar. 13, 2025

(54) DISPENSING SYSTEM

(71) Applicant: BJMC Investment Series LLC, Rockwall, TX (US)

(72) Inventor: Minhchau Le, Rockwall, TX (US)

(73) Assignee: BJMC Investment Series LLC, Rockwall, TX (US)

(21) Appl. No.: 18/463,823

(22) Filed: Sep. 8, 2023

(51) Int. Cl. A61J 7/04; A61J 7/00; G07C 9/25; G16H 10/60

(52) U.S. Cl. CPC A61J 7/0481; A61J 7/0084; A61J 7/0454; G07C 9/257; G16H 10/60; A61J 2200/30; A61J 2200/70; A61J 2200/72

(57) ABSTRACT

A dispensing system is disclosed. The dispensing system may include a plurality of compartments configured to store medicines associated with a patient. The dispensing system may further include a medicine management unit that may include a transceiver and a processor. The transceiver may be configured to receive a medicine delivery schedule associated with the patient. The processor may be configured to obtain the medicine delivery schedule from the transceiver and identify a medicine and a medicine delivery time. The processor may be further configured to output a notification at the medicine delivery time to indicate to the patient to collect the medicine. In addition, the processor may be further configured to determine that the patient is in proximity to a dispensing system front portion responsive to outputting the notification and cause a compartment to move from a closed position to an open position.

### TECHNICAL FIELD

The present disclosure relates to a dispensing system, and more specifically to a medicine dispensing system that automatically dispenses medicines based on medicine schedule.

### BACKGROUND

Elderly people are typically not able to recall the medicines that they need to take, respective medicine time, prescribed medicine dose, etc. Therefore, elderly people generally rely on external assistance (e.g., caregivers, family members, etc.) to track and provide the medicines. There may be instances when the caregivers may not be available, which may result in the elderly people missing their medicines.

There exist conventional medicine management devices that facilitate elderly people to take their medicines. However, the conventional medicine management devices may not operate for different types of medicines and hence may not be convenient to use by the elderly people.

Thus, there is a need for a system that operates for different types of medicines and facilitates an elderly person to track and take medicines on time.

It is with respect to these and other considerations that the disclosure made herein is presented.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIG. 1 depicts an example environment in which techniques and structures for providing the systems and methods disclosed herein may be implemented.

FIG. 2 depicts a block diagram of an example dispensing system in accordance with the present disclosure.

FIG. 3 depicts an isometric view of the dispensing system of FIG. 2 in accordance with the present disclosure.

FIGS. 4, 5, 6 and 7 provide example snapshots of a patient operating the dispensing system of FIG. 2 in accordance with the present disclosure.

FIG. 8 depicts a flow diagram of an example method for dispensing medicine from the dispensing system, in accordance with the present disclosure.

### DETAILED DESCRIPTION

### Overview

The present disclosure is directed towards a dispensing system that may be configured to dispense medicines based on a medicine delivery schedule associated with a patient. The dispensing system may be configured to store different types of medicines (such as tablets, capsules, eye-drops etc.) in a plurality of compartments of the dispensing system. The dispensing system may include a medicine management unit (“unit”) that may be configured to automatically dispense a medicine from the dispensing system based on medicine's delivery schedule.

In some aspects, the unit may obtain the medicine's delivery schedule from a user device associated with a caregiver or any external device. Responsive to obtaining the medicine delivery schedule, the unit may identify a medicine to be provided to the patient and a corresponding medicine delivery time based on the medicine delivery schedule. When the medicine delivery time arrives, the unit may output a notification to the patient to indicate to the patient to collect the medicine from the dispensing system. The unit may output the notification in any form including, but not limited to, an audible signal, haptic feedback (e.g., via a smart device worn by the patient such as a smart wristband), a visual indication, and/or the like.

In further aspects, the unit may determine whether the patient has arrived in proximity to the dispensing system to take the medicine. In an exemplary aspect, the unit may perform such determination responsive to outputting the notification. Responsive to a determination that patient may have arrived in proximity to the dispensing system, the unit may perform patient authentication to ensure that the medicine is given to an intended person. In some aspects, the unit may perform the authentication via face recognition technology. The unit may be configured to cause a compartment (having the medicine) to slide out (e.g., move from a closed position to an open position) from a dispensing system body when the patient may be authenticated. Responsive to the compartment sliding out, the patient may pick the medicine from the compartment.

In further aspects, the unit may be configured to provide instructions (associated with medicine consumption process) to the patient when the compartment slides out. The instructions may include a way to take the medicine, medicine dosage, and/or the like. In some aspects, the unit may fetch a pre-stored video/audio (e.g., a video message from the caregiver) from a system memory and output the video/audio to enable the patient to conveniently take the medicine. The patient may view/hear the instructions and may take the medicine and close the compartment.

In further aspects, the unit may determine whether the patient has closed the compartment and may determine that the patient may have successfully taken the medicine. In some aspects, based on a determination that the patient may not have taken the medicine, the unit may transmit a notification to the caregiver.

In additional aspects, the dispensing unit may include a temperature unit that may be configured to control temperature of one or more compartments in the dispensing system. The unit may be configured to obtain a desired temperature associated with the compartments and may provide instruction to the temperature unit to maintain the compartment temperature accordingly. The temperature unit may be further configured to determine a current temperature of a compartment and display the current temperature on a compartment front portion/display screen or in proximity to the compartment.

The present disclosure discloses a dispensing system that facilitates the patient to take the medicines by own, without relying on external assistance. In addition, the dispensing system may output reminders to the patient based on the medicine delivery schedule, and the patient may not have to recall medicine schedule and medicine dosage. The dispensing system further facilitates storing different types of medicines in a single dispensing system and may also maintain temperature of respective medicines.

These and other advantages of the present disclosure are provided in detail herein.

### Illustrative Embodiments

The disclosure will be described more fully hereinafter with reference to the accompanying drawings, in which example embodiments of the disclosure are shown, and not intended to be limiting.

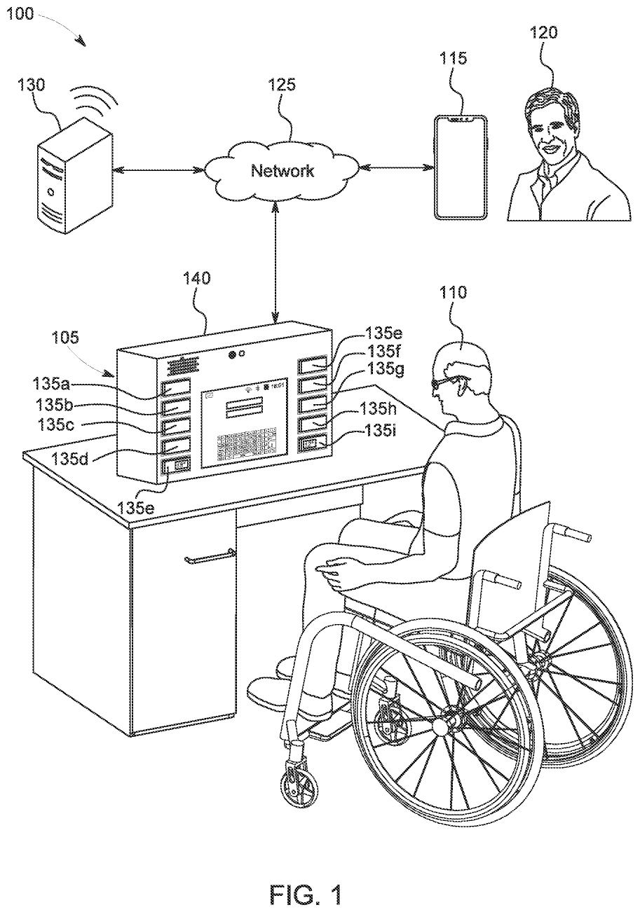

FIG. 1 depicts an example environment 100 in which techniques and structures for providing the systems and methods disclosed herein may be implemented. The environment 100 may include a dispensing system 105 that may enable a patient 110 to take medicines based on prescribed medicine schedule. The dispensing system 105 may be a medicine dispensing system that may be configured to enables the patient 110 to take medicines of different types, e.g., capsules, liquids, a cream/gel, eye-drops, and/or the like. The dispensing system 105 may be communicatively coupled to a user device 115 that may be associated with a caregiver 120, via a network 125. In addition, the dispensing system 105 may be communicatively coupled to an external server 130 via the network 125. In some aspects, the server 130 may be associated with a service provider managing the dispensing system 105, or the server 130 may be associated with a medical facility such as a hospital or a clinic. Further, the user device 115 may be, for example, a mobile phone, a laptop, a computer, a tablet, or any other similar device with communication capabilities.

The network 125 may be, for example, a communication infrastructure in which the connected devices discussed in various embodiments of this disclosure may communicate. The network 125 may be and/or include the Internet, a private network, public network or other configuration that operates using any one or more known communication protocols such as, for example, transmission control protocol/Internet protocol (TCP/IP), Bluetooth®, BLE®, Wi-Fi based on the Institute of Electrical and Electronics Engineers (IEEE) standard 802.11, UWB, and cellular technologies such as Time Division Multiple Access (TDMA), Code Division Multiple Access (CDMA), High Speed Packet Access (HSPDA), Long-Term Evolution (LTE), Global System for Mobile Communications (GSM), and Fifth Generation (5G), to name a few examples.

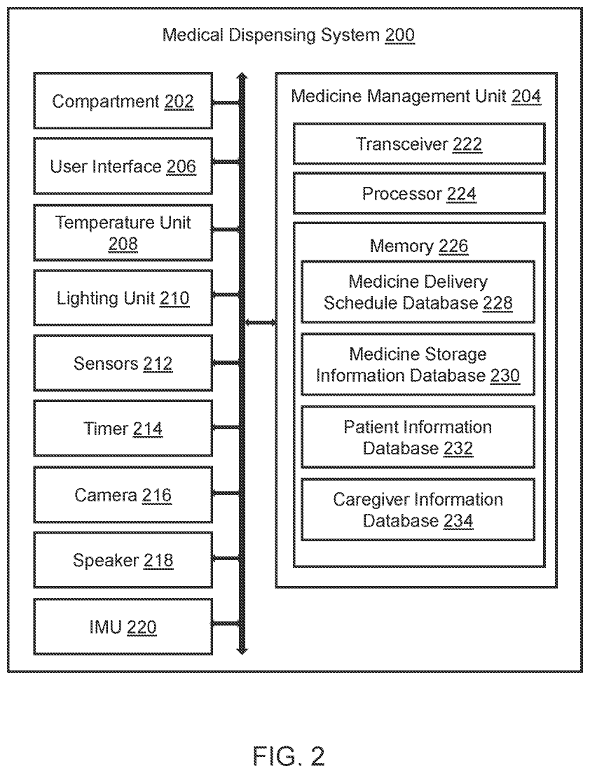

The dispensing system 105 may include a plurality of compartments 135a-i (collectively referred as a plurality of compartments 135) that may be configured to stored medicines associated with the patient 110. For example, the compartments 135 may store tablets, capsules, eye drops, a cream/gel, and/or the like. In some aspects, each compartment may be shaped as a cube or a cuboid having an enclosure in which the medicines may be placed or stored. The dispensing system 105 may further include a medicine management unit or “unit” (shown as medicine management unit 204 in FIG. 2) that may be communicatively coupled with the compartments 135.

In some aspects, the unit may be configured to obtain medicine delivery schedule for the patient 110 from the user device 115 associated with the caregiver 120 or from the server 130. Responsive to obtaining the medicine delivery schedule, the unit may be configured to identify a medicine that may be required to be provided to the patient 110 and corresponding medicine delivery time based on the obtained medicine delivery schedule. When the medicine delivery time arrives, the unit may be configured to output a notification to indicate to the patient 110 to collect the medicine. The unit may output the notification at the medicine delivery time. The unit may output the notification in any form including, but not limited to, an audible signal, haptic feedback, a visual indication, and/or the like. In some aspects, the notification may also include instructions on the way to take the medicine and/or the prescribed dosage. For example, the unit may output a notification instructing the patient to take two drops of an eye-drop in each eye at the medicine delivery time. In this manner, the unit provides reminders to the patient 110 so that the patient 110 may take the required medicines on scheduled times.

In further aspects, the unit may be configured to determine whether the patient 110 has arrived in proximity to the dispensing system 105 to take the medicine, responsive to hearing/viewing the notification. Stated another way, the unit may determine whether the patient 110 has arrived in proximity to a dispensing system front portion (e.g., by obtaining inputs from a system camera) responsive to the unit outputting the notification. When the unit determines that the patient 110 may be present in proximity to the dispensing system front portion, the unit may cause a compartment (having the identified medicine) to move from a closed position to an open position to enable the patient 110 to take the medicine. In some aspects, the compartment may be in the closed position when the compartment may be disposed in an interior portion of a body 140 of the dispensing system 105, and the compartment may be in the open position when the compartment may be disposed partially or fully outside of the body 140. The unit may cause the compartment to move from the closed position to the open position by sliding out the compartment from inside the body 140 to outside. Responsive to the compartment being outside of the body 140, the patient 110 may take out the medicine (e.g., the eye-drop) from the compartment. Further, the patient 110 may put the eye-drop back in the compartment after taking the prescribed drops and close the compartment back to the closed position. In some aspects, the unit may cause the compartment to move automatically from the open position to the closed position when the unit determines that the patient 110 may have taken the medicine (e.g., using the system camera/sensors).

On the other hand, when the unit determines that the patient 110 has not arrived in proximity to the dispensing system front portion for a predefined time duration after the unit outputs the notification, the unit may transmit an alert notification to the user device 115 and/or the server 130. Thereafter, the caregiver 120 and/or a server operator may take one or more remedial actions.

Further details of the dispensing system 105 are described below in conjunction with subsequent figures.

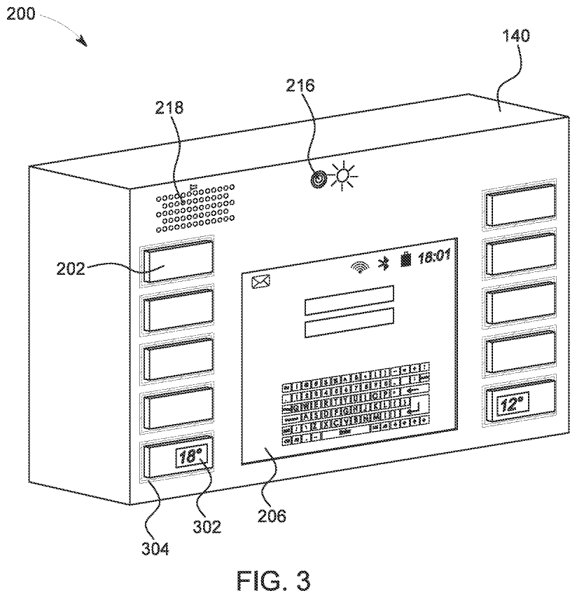

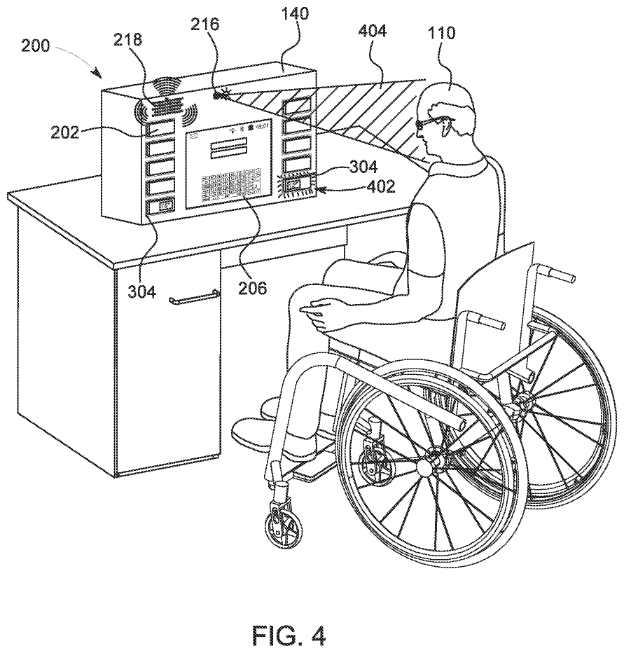

FIG. 2 depicts a block diagram of an example dispensing system 200 in accordance with the present disclosure. FIG. 2 will be described in conjunction with FIGS. 3-7. FIG. 3 depicts an isometric view of the dispensing system 200. FIGS. 4-7 provide example snapshots of a patient operating the dispensing system 200 in accordance with the present disclosure.

The dispensing system 200 may be same as the dispensing system 105 described above in conjunction with FIG. 1. The dispensing system 200 may include a plurality of compartments 202 (same as the compartments 135) that may be configured to store medicines associated with a patient (e.g., the patient 110). The medicines may include tablets, capsules, eye drops, a cream/gel, and/or the like. The compartments 202 may be of any shape, size, and design, which may be based on the size and design of the dispensing system 200. In an exemplary aspect, each compartment 205 may be shaped as a cuboid having an enclosure in a compartment interior portion, in which the medicines may be stored. The compartments 202 may be disposed in the dispensing system 200 in any location/pattern. For example, the compartments 202 may be disposed vertically in proximity to dispensing system side edges, as depicted in FIG. 3. In other aspects (not shown), the compartments 202 may be disposed horizontally in proximity to dispensing system top/bottom edges or in any other position, without departing from the present disclosure scope. The designs and sizes of different compartments may be same or different.

The dispensing system 200 may further include a plurality of components including, but not limited to, a medicine management unit 204 (or unit 204), a user interface 206, a temperature unit 208, a lighting unit 210, one or more sensors 212, a timer 214, a camera 216, a speaker 218, an inertial measurement unit (IMU) 220, and/or the like. In some aspects, the dispensing system 200 may include an integrated power supply such as battery to operate different components of the dispensing system 200 (e.g., during power outage). In further aspects, the dispensing system 200 may include braille labels to assist visually impaired patients. The unit 204 may include one or more components including, but not limited to, a transceiver 222, a processor 224, and a memory 226. The transceiver 222 may be configured to transmit/receive signals/data/information to/from external systems or devices (e.g., the user device 115, server 130, etc.), via the network 125.

In some aspects, the memory 226 may store programs in code and/or store data for performing various system operations in accordance with the present disclosure. Specifically, the processor 224 may be configured and/or programmed to execute computer-executable instructions stored in the memory 226 for performing various system functions in accordance with the disclosure. Consequently, the memory 226 may be used for storing code and/or data code and/or data for performing operations in accordance with the present disclosure.

In one or more aspects, the processor 224 may be disposed in communication with one or more memory devices (e.g., the memory 226 and/or one or more external databases (not shown in FIG. 2)). The memory 226 can include any one or a combination of volatile memory elements (e.g., dynamic random-access memory (DRAM), synchronous dynamic random-access memory (SDRAM), etc.) and can include any one or more nonvolatile memory elements (e.g., erasable programmable read-only memory (EPROM), flash memory, electronically erasable programmable read-only memory (EEPROM), programmable read-only memory (PROM), etc.).

The memory 226 may be one example of a non-transitory computer-readable storage medium and may be used to store programs in code and/or to store data for performing various operations in accordance with the present disclosure. The instructions in the memory 226 may include one or more separate programs, each of which may include an ordered listing of computer-executable instructions for implementing logical functions.

In some aspects, the memory 226 may include a plurality of databases including, but not limited to, a medicine delivery schedule database 228, a medicine storage information database 230, a patient information database 232, a caregiver information database 234, and/or the like. The medicine delivery schedule database 228 may store medicine delivery schedule information associated with a patient (e.g., the patient 110). The medicine delivery schedule information may include medicine names, corresponding prescribed medicine delivery times, dosage, etc. For example, the medicine delivery schedule information may indicate that tablet “A” needs to be given to the patient 110 three times in a day (e.g., at 8 AM, 2 PM, and 8 PM). In another example, the medicine delivery schedule information may indicate that eye drop “B” needs to be given to the patient 110 at 9 AM and the patient 110 may be required to put three drops in each eye. The unit 204 may obtain such medicine delivery schedule information from the user device 115 and/or the server 130, as described above.

The medicine storage information database 230 may store a mapping of the medicines and the compartments 202. The mapping may indicate which medicine is stored in which compartment. For example, the mapping may indicate that the tablet “A” may be stored in a compartment “A” (from the plurality of compartments 202) and eye drop “B” may be stored in a compartment “B” (from the plurality of compartments 202). In some aspects, the caregiver 120 may pre-store the medicines in respective compartments and may transmit the mapping to the unit 204 (for storage in the medicine storage information database 230) via the user device 115.

The patient information database 232 may store patient information associated with the patient 110. The patient information may include, for example, patient name, patient age, patient medical history, and/or the like. In some aspects, the caregiver 120 may transmit the patient information to the unit 204 (for storage in the patient information database 232) via the user device 115. Similarly, the caregiver information database 234 may store include information associated with the caregiver 120 including, but not limited to, a caregiver name, a caregiver contact information such as a phone number, a caregiver relationship with the patient 110, and/or the like. In some aspects, the caregiver information database 234 may further store audio or video message(s) of the caregiver 120 which may enable the patient 110 to take a specific medicine. As an example, the audio or video message may include a procedure/instruction to take the specific medicine.

In some aspects, the temperature unit 208 may be configured to control temperature of one or more compartments (from the plurality of compartments 202) and maintain compartment temperature at a desired temperature. For example, if a specific medicine stored in a compartment requires cold temperature, e.g., a temperature below room temperature, the temperature unit 208 may regulate and maintain temperature of the compartment storing the specific medicine at the desired temperature. In some aspects, the temperature unit 208 may be configured to obtain instructions from the unit 204 and control temperature of respective compartments based on the instructions. For example, the temperature unit 208 may maintain cool environment for a compartment in which eye drop may be stored. In some aspects, the temperature unit 208 may be further configured to determine a current temperature of a compartment and may display the temperature on a compartment display screen 302 (as depicted in FIG. 3) associated with the compartment.

The lighting unit 210 may include a plurality of LEDs 304 (shown in FIG. 3) that may be disposed in proximity to (e.g., at a periphery of) or at each compartment 202. The LEDs 304 may illuminate to provide visual indication to the patient 110 to take medicine from the illuminated compartment including the LEDs 304. The lighting unit 210 may illuminate the LEDs 304 in a predetermined pattern, responsive to receiving command signals from the unit 204. The predetermined pattern may include illuminating the LEDs 304 in a moving pattern, in a single color with a predefined intensity, illuminating specific/selective portions of the compartment 202, and/or the like.

The sensors 212 may include, for example, proximity sensors, weight sensors, and/or the like. In some aspects, the sensors 212 may monitor whether a compartment may be empty or may include medicines. The unit 204 may obtain inputs from the sensors 212 and may provide indication to the caregiver 120 to refill the compartment(s). The timer 214 may be configured to set reminders for medicine delivery. Specifically, the timer 214 may be configured to obtain the medicine delivery schedule (including the medicine delivery time) and may set reminders to enable the unit 204 to output the notification at the medicine delivery time. The user interface 206 may be configured to receive inputs from the patient 110 and/or the caregiver 120 and may display notifications/other inputs to the patient 110 and/or the caregiver 120.

The camera 216 may be disposed at a front portion of a body 140 of the dispensing system 200. The camera 216 may be configured to capture images associated with the patient 110, when the patient 110 may be disposed in proximity to the dispensing system front portion. The speaker 218 may be configured to audibly output notifications, commands, etc. The IMU 220 may be configured to determine whether a compartment is in the closed position or the open position, as described later below.

In operation, the transceiver 222 may receive the medicine delivery schedule from the user device 115 associated with the caregiver 120 and/or the server 130 and may store the medical delivery schedule in the medicine delivery schedule database 228. The processor 224 may obtain the medicine delivery schedule from the medicine delivery schedule database 228 or directly from the transceiver 222. Responsive to obtaining the medicine delivery schedule, the processor 224 may identify a medicine to be given to the patient 110 along with the corresponding medicine delivery time based on the medicine delivery schedule. For example, the processor 224 may determine that a medicine “A” (e.g., an eye-drop) needs to be given to the patient 110 every day at 10 AM. In addition, the processor 224 may identify (or select) a compartment 402 (depicted in FIG. 4), from the plurality of compartments 202, in which the caregiver 120 may have pre-stored the medicine “A”. In some aspects, to identify the compartment 402, the processor 224 may fetch the mapping of the medicines and the compartments 202 from the memory 226 (e.g., from the medicine storage information database 230), and identify the compartment 402 based on the mapping.

At the scheduled medicine delivery time, the timer 214 may transmit a trigger signal to the processor 224. Responsive to obtaining the trigger signal from the timer 214, the processor 224 may output a notification to indicate to the patient 110 that it is time to collect/take the medicine. The processor 224 may output the notification in a preset manner. For example, the processor 224 may output a series of notifications for a predefined time duration at a predefined frequency until the patient 110 arrives in proximity to the dispensing system 200 to collect/take the medicine. Alternatively, the processor 224 may output a single notification for the predefined time duration. In other aspects, the processor 224 may output the notification based on patient and/or caregiver preferences.

In some aspects, the processor 224 may output an audio signal, via the speaker 218, as the notification for the patient 110. The audio signal may include, for example, a reminder tone, a preset message recorded by the caregiver 120, a preset music, and/or the like. In further aspects, the processor 224 may output a visual notification for the patient 110. For example, the processor 224 may activate the LEDs 304 (via the lighting unit 210) associated with the selected compartment 402 in a predetermined pattern to visually indicate the compartment 402 in which the medicine may be stored. In some aspects, the processor 224 may activate the LEDs 304 that are only associated with the selected compartment 402, and not LEDs associated with other compartments. The predetermined pattern may include illuminating the LEDs 304 associated with the compartment 402 in a moving pattern, flashing the LEDs 304 in a single color with a predefined intensity, illuminating specific/selective portions of the compartment 402, illuminating the compartment periphery, and/or the like. In further aspects, the processor 224 may transmit the notification, via the transceiver 222, to a user device associated with the patient 110. The patient 110 may arrive in proximity to the dispensing system 200 responsive to hearing/viewing the notification.

The processor 224 may be configured to determine whether the patient 110 may have arrived in proximity to the dispensing system front portion, responsive to outputting the notification. In some aspects, the processor 224 may make this determination based on images obtained from the camera 216. An example field of view (FOV) of the camera 216 is depicted as FOV 404 in FIG. 4. Responsive to determining that the patient 110 may not have arrived in proximity to the dispensing system front portion for a predefined time duration when the processor 224 outputs the notification, the processor 224 may transmit, via the transceiver 222, an alert signal/notification to the caregiver 120 and/or the server 130. In this case, the caregiver 120 may call the patient 110 (e.g., via a user device associated with the patient 110) and request the patient 110 to take the medicines.

On the other hand, responsive to determining that the patient 110 may have arrived in proximity to the dispensing system front portion, the processor 224 perform patient authentication. Specifically, the processor 224 may obtain patient images using the camera 216 when the patient 110 may be present in proximity to the dispensing system front portion and may compare the captured images with pre-stored patient images. Specifically, the processor 224 may fetch pre-stored patient images from the memory 226 (e.g., from the patient information database 232), and compare the fetched images with the images obtained from the camera 216. The processor 224 may perform the patient authentication based on the image comparison. In this case, the processor 224 authenticates the patient 110 using facial recognition. In further aspects, the processor 224 may authenticate the patient 110 by using any other authentication technology, e.g., iris recognition, fingerprint scanning, and/or the like. A person ordinarily skilled in the art may appreciate that the dispensing system 200 performs the patient authentication to ensure that the dispensing system 200 is not misused, and the medicine is provided to the patient (e.g., the patient 110) who is actually the intended medicine recipient.

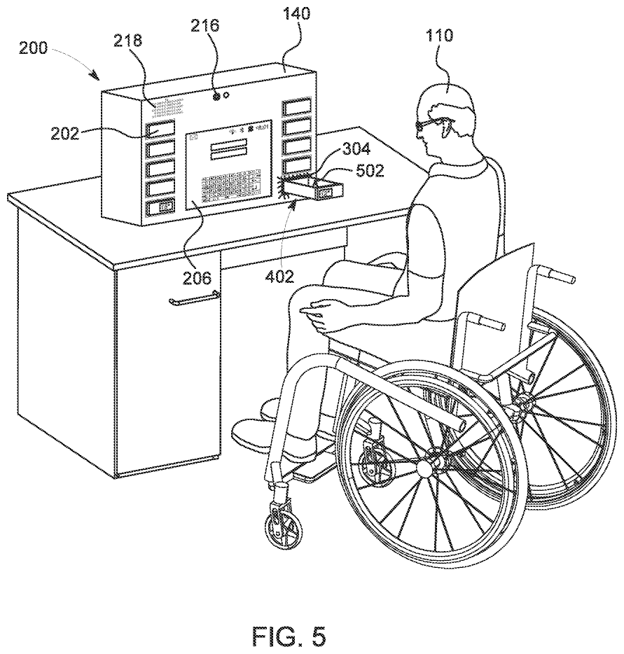

Responsive to successful patient authentication (i.e., responsive to determining that patient 110 is the intended medicine recipient), the processor 224 may cause the compartment 402 to move from a closed position to an open position (or slide out), as depicted in FIG. 5. In some aspects, the compartment 402 may be disposed in an interior portion of the body 140 (or dispensing system interior portion) when the compartment 402 may be in the closed position, and may be at least partially disposed out of the body 140 (or medical dispensing system interior portion) in the open position. Specifically, the processor 224 may slide out the compartment 402 from the dispensing system front surface while moving the compartment 402 from the closed position to the open position, to enable the patient 110 to conveniently take out a medicine 502 from the compartment 402, as shown in FIG. 5. In an exemplary aspect, the medicine 502 may be an eye-drop, as depicted in FIG. 5. Since the compartment 402 slides out from the body 140, the eye-drops do not get spilled in the compartment enclosure. In other aspects, the medicine 502 may be a capsule, a tablet, a cream/gel, and/or the like.

In some aspects, the compartment 402 may not slide out, and may be configured to drop down the medicine (e.g., tablet, capsule etc., when the patient 110 is ready to collect the medicine) to a bottom tray accessible to the patient 110. For example, the compartment 402 (or the dispensing system 200) may include a mechanism to move the scheduled medicine to the bottom tray and may prevent the tipping over of the compartment 402.

In further aspects, the processor 224 may be configured to obtain medicine delivery instructions associated with the medicine 502 from the memory 226 (e.g., from the medicine delivery schedule database 228, or the caregiver information database 234 which may pre-store the instructions as audio or video message(s) from the caregiver 120). Responsive to obtaining the medicine delivery instructions, the processor 224 may output the instructions from the speaker 218 or the user interface 206. In some aspects, the processor 224 may output the instructions for the patient 110 when the compartment 402 slides out (e.g., moves from the closed position to the open position). The instructions may be associated with the medicine 502 to be delivered to the patient 110.

In some aspects, the processor 224 may identify a medicine dosage based on the medicine delivery instructions, and may output a notification for the patient 110 based on the medical dosage identification. The notification may indicate the appropriate medical dosage associated with the medicine 502 that the patient 110 may take. For example, the processor 224 may output a notification to the patient 110 indicating to take out the medicine 502 from the compartment 402 and put 2 drops in each eye. The notification may further include instructions or request to put back the medicine 502 in the compartment 402 after taking the dosage, and close the compartment 402 (e.g., move or slide-in the compartment 402 from the open position to the closed position).

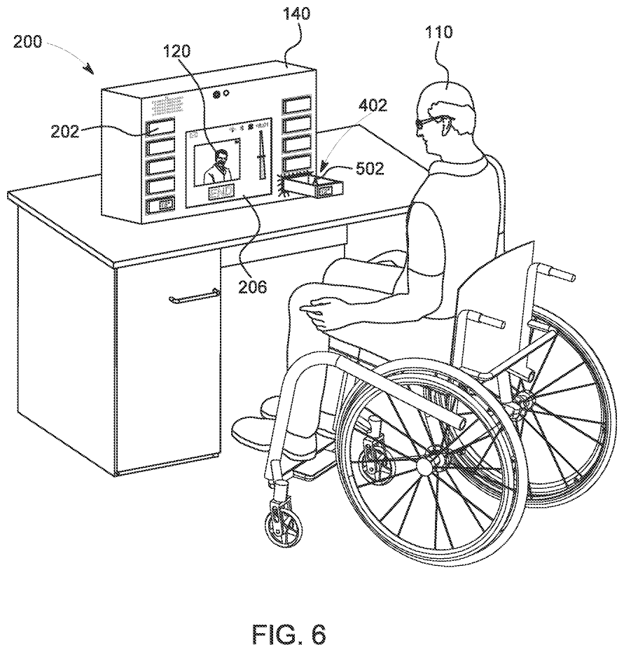

As described above, the processor 224 may obtain inputs (e.g., audio and/or video messages) from the user device 115 associated with the caregiver 120 and may output notifications/instructions based on the inputs. In some aspects, the processor 224 may output the notification to the patient 110 when the compartment 402 slides out. In this case, the processor 224 may obtain a pre-stored video (e.g., a video message) from the memory 226 (e.g., from the caregiver information database 234) when the compartment 402 slides out and may output the pre-stored video on the user interface 206, as depicted in FIG. 6. The pre-stored video may include a video message that may include instructions on best or appropriate way to take the medicine 502. The instructions may be associated with the medicine 502 to be delivered to the patient 110 and may be different for different medicines. processor 224A person ordinarily skilled in the art may appreciate that displaying the video message on the user interface 206 enables the patient 110 to understand the instructions clearly from the caregiver 120, and then appropriately and safely take the medicine 502. In further aspects, the processor 224 may obtain a pre-stored audio message from the memory 226 and may output the audio message via the speaker 218 to enable the patient 110 to conveniently take the medicine 502. In addition, the processor 224 may facilitate the patient 110 to connect with the caregiver 120 by using the dispensing system 200, for example, via a video/audio call (e.g., using face time technology).

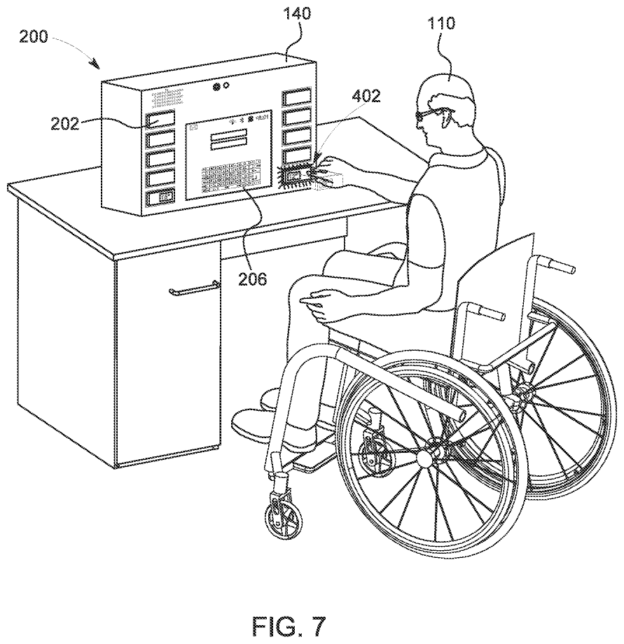

Responsive to hearing/viewing the instructions, the patient 110 may take the medicine 502 and place the remaining medicine (if required) back in the compartment 402. The patient 110 may then move the compartment 402 from the open position to the closed position, as shown in FIG. 7. Stated another way, the patient 110 may slide the compartment 402 back into the body 140, when the patient 110 may have taken the medicine 502.

The processor 224 may be configured to determine whether the patient 110 may have closed the compartment 402. Stated another way, the processor 224 may be configured to determine whether the patient 110 may have moved the compartment 402 from the open position to the closed position. In this case, the processor 224 may obtain inputs from the IMU 220 and determine whether the patient 110 has closed the compartment 402 based on the inputs from the IMU 220. In some aspects, the processor 224 may determine whether the patient 110 may have taken the medicine 502 based on a determination that the patient 110 may have closed the compartment 402 or not. In some aspects, the patient 110 may tap the compartment 402 (or any other portion of the dispensing system 200) to indicate that the patient 110 has taken the medicine.

In some aspects, the processor 224 may determine that the patient 110 may not have taken the medicine 502 when the processor 224 determines that the patient 110 has not moved the compartment 402 from the open position to the closed position. For example, the processor 224 may determine that the patient 110 may not have taken the medicine 502 when the processor 224 determines that the compartment 402 is not closed after 5 mins (or after a predefined time duration) of opening the compartment 402. In such scenarios, the processor 224 may provide another instruction/notification to the patient 110 to ensure that the patient 110 takes the medicine 502 and closes the compartment 402. In further aspects, the processor 224 may transmit a notification to the user device 115 to indicate that the patient 110 may not have taken the medicine 502. The caregiver 120 may view the notification on the user device 115 and perform appropriate actions to ensure that the patient 110 takes the medicine 502.

On the other hand, responsive to determining that the patient 110 may have moved the compartment 402 from the open position to the closed position, and hence may have taken the medicine 502, the processor 224 may output a predefined message (e.g., “Thank You”) via the user interface 206 or the speaker 218.

In some aspects, the processor 224 may be further configured to track whether the patient 110 may have taken the medicine 502 as per the medicine delivery schedule or missed taking the medicine 502. The processor 224 may transmit, via the transceiver 222, such tracking information to the user device 115 so that the caregiver 120 may be made aware. In further aspects, the processor 224 may determine if there are no medicines in one or more compartments (e.g., via the sensors 212) or the compartments may be vacant and may transmit a notification to the user device 115 to refill the determined compartments.

In additional aspects, the processor 224 may be configured to obtain desired temperature of one or more compartments (e.g., via the transceiver 222) from the server 130 and/or the user device 115. For example, the processor 224 may obtain the desired compartment temperature from the caregiver 120 via the user device 115 or the user interface 206. The desired temperature may be associated with a medicine (e.g., the medicine 502) stored in a compartment (e.g., the compartment 402). For example, the processor 224 may obtain the desired temperature for the medicine 502 stored in the compartment 402 from the caregiver 120. Responsive to obtaining the desired temperature, the processor 224 may transmit an instruction to the temperature unit 208 to maintain the desired temperature in the compartment 402. The temperature of different compartments may be same or different from each other. In further aspects, the processor 224 may obtain a current temperature of one or more compartments from the temperature unit 208 and may cause to display the current temperature. In some aspects, the processor 224 may cause to display the current temperature on respective compartment display screens (e.g., on a compartment front surface). In further aspects, the processor 224 may cause to display the current temperature on any other location, e.g., the user interface 206.

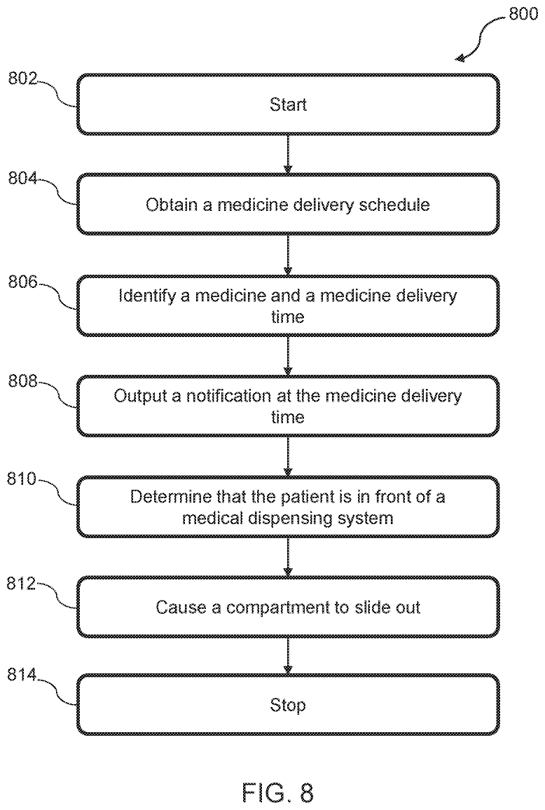

FIG. 8 depicts a flow diagram of an example method 800 for dispensing medicine from the dispensing system 200, in accordance with the present disclosure. FIG. 8 may be described with continued reference to prior figures. The following process is exemplary and not confined to the steps described hereafter. Moreover, alternative embodiments may include more or less steps than are shown or described herein and may include these steps in a different order than the order described in the following example embodiments.

Referring to FIG. 8, at step 802, the method 800 may commence. At step 804, the method 800 may include obtaining, by the processor 224, a medicine delivery schedule associated with a patient (e.g., the patient 110). As described above, the processor 224 may obtain the medical delivery schedule from the user device 115 or the server 130, via the transceiver 222.

At step 806, the method 800 may include identifying, by the processor 224, a medicine and a medicine delivery time based on the medicine delivery schedule. At step 808, the method 800 may include outputting, by the processor 224, a notification at the medical delivery time to indicate to the patient 110 to collect/take the medicine from the dispensing system 200.

At step 810, the method 800 may include determining, by the processor 224, that the patient 110 may be in proximity to a dispensing system front portion. In some aspects, the processor 224 may perform the determination after outputting the notification to the patient 110.

At step 812, the method 800 may include causing, by the processor 224, a compartment (having the medicine) to move from a closed position to an open position, as described above. Stated another way, the processor 224 may cause the compartment to slide out from the body 140 when the patient 110 may be present in proximity to the dispensing system front portion. The method 800 may end at step 814.

In the above disclosure, reference has been made to the accompanying drawings, which form a part hereof, which illustrate specific implementations in which the present disclosure may be practiced. It is understood that other implementations may be utilized, and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a feature, structure, or characteristic is described in connection with an embodiment, one skilled in the art will recognize such feature, structure, or characteristic in connection with other embodiments whether or not explicitly described.

It should also be understood that the word “example” as used herein is intended to be non-exclusionary and non-limiting in nature. More particularly, the word “example” as used herein indicates one among several examples, and it should be understood that no undue emphasis or preference is being directed to the particular example being described.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating various embodiments and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their ordinary meanings as understood by those knowledgeable in the technologies described herein unless an explicit indication to the contrary is made herein. In particular, use of the singular articles such as “a,” “the,” “said,” etc., should be read to recite one or more of the indicated elements unless a claim recites an explicit limitation to the contrary. Conditional language, such as, among others, “can,” “could,” “might,” or “may,” unless specifically stated otherwise, or otherwise understood within the context as used, is generally intended to convey that certain embodiments could include, while other embodiments may not include, certain features, elements, and/or steps. Thus, such conditional language is not generally intended to imply that features, elements, and/or steps are in any way required for one or more embodiments.

That which is claimed is:

1. A dispensing system comprising:
a plurality of compartments configured to store medicines associated with a patient; and
a medicine management unit communicatively coupled to the plurality of compartments, wherein the medicine management unit comprises:
a transceiver configured to receive a medicine delivery schedule associated with the patient; and
a processor communicatively coupled to the transceiver, wherein the processor is configured to:
obtain the medicine delivery schedule from the transceiver;
identify a medicine and a medicine delivery time based on the medicine delivery schedule;
output a notification at the medicine delivery time to indicate to the patient to collect the medicine;
determine that the patient is in proximity to a dispensing system front portion responsive to outputting the notification; and
cause a compartment, of the plurality of compartments, to move from a closed position to an open position to enable the patient to take the medicine responsive to determining that the patient is in proximity to the dispensing system front portion.

2. The dispensing system of claim 1, wherein the compartment is disposed in a dispensing system interior portion in the closed position, wherein the compartment is at least partially disposed out of the dispensing system interior portion in the open position, and wherein the processor causes the compartment to move from the closed position to the open position by causing the compartment to slide out from the dispensing system interior portion.

3. The dispensing system of claim 1 further comprising a memory configured to store a mapping of a plurality of medicines with the plurality of compartments, and wherein the processor is further configured to:
fetch the mapping from the memory;
select the compartment from the plurality of compartments based on the mapping, wherein the compartment stores the medicine; and
cause the compartment to move from the closed position to the open position responsive to selecting the compartment.

4. The dispensing system of claim 1, wherein the processor is further configured to:
obtain patient images responsive to the determination that the patient is in proximity to the dispensing system front portion;
perform patient authentication based on the patient images; and
cause the compartment to move from the closed position to the open position when the patient is authenticated.

5. The dispensing system of claim 1, wherein the processor outputs the notification by outputting an audible signal.

6. The dispensing system of claim 5, wherein the processor further outputs the notification by activating lights associated with the compartment in a predetermined pattern.

7. The dispensing system of claim 1, wherein the processor is further configured to:
obtain a pre-stored video; and
cause to display the pre-stored video on a dispensing system user interface to enable the patient to take the medicine.

8. The dispensing system of claim 1 further comprising an inertial measurement unit, wherein the processor is further configured to:
obtain inputs from the inertial measurement unit;
determine that the patient has moved the compartment from the open position to the closed position based on the inputs obtained from the inertial measurement unit; and
determine that the patient has taken the medicine based on a determination that the patient has moved the compartment from the open position to the closed position.

9. The dispensing system of claim 8, wherein the processor is further configured to:
determine that the patient has not taken the medicine based on a determination that the patient has not moved the compartment from the open position to the closed position; and
transmit a notification to a caregiver user device to indicate that the patient has not taken the medicine.

10. The dispensing system of claim 9, wherein the processor is further configured to:
receive inputs from the caregiver user device; and
output a second notification responsive to receiving the inputs from the caregiver user device.

11. The dispensing system of claim 1 further comprising a temperature unit configured to control temperature of one or more compartments from the plurality of compartments.

12. The dispensing system of claim 11, wherein the processor is further configured to:
obtain desired temperature of the one or more compartments; and
transmit an instruction to the temperature unit to control temperature of the one or more compartments based on the desired temperature.

13. The dispensing system of claim 11, wherein the processor is further configured to:
obtain a current temperature of the one or more compartments; and
cause to display the current temperature on respective compartment display screens responsive to obtaining the current temperature.

14. The dispensing system of claim 1, wherein the processor is further configured to:
identify a medicine dose based on the medical delivery schedule; and
output the notification based on the identification of the medicine dose, wherein the notification indicates the medicine dose to be taken by the patient.

15. A method to dispense medicines from a dispensing system, the method comprising:
obtaining, by a processor, a medicine delivery schedule associated with a patient;
identifying, by the processor, a medicine and a medicine delivery time based on the medicine delivery schedule;
outputting, by the processor, a notification at the medicine delivery time to indicate to the patient to collect the medicine;
determining, by the processor, that the patient is in proximity to a dispensing system front portion responsive to outputting the notification; and
causing, by the processor, a compartment of a plurality of compartments to move from a closed position to an open position to enable the patient to take the medicine responsive to determining that the patient is in proximity to the dispensing system front portion, wherein the plurality of compartments is configured to store medicines associated with the patient.

16. The method of claim 15, wherein the compartment is disposed in a dispensing system interior portion in the closed position, wherein the compartment is at least partially disposed out of the dispensing system interior portion in the open position, and wherein causing the compartment to move from the closed position to the open position comprises causing the compartment to slide out from the dispensing system interior portion.

17. The method of claim 15 further comprising:
fetching a mapping of a plurality of medicines with the plurality of compartments from a memory;
selecting the compartment from the plurality of compartments based on the mapping; and
causing the compartment to move from the closed position to the open position responsive to selecting the compartment.

18. The method of claim 15, wherein outputting the notification comprises activating lights associated with the compartment in a predetermined pattern.

19. The method of claim 15, wherein outputting the notification comprises outputting an audible signal.

20. A non-transitory computer-readable storage medium in a distributed computing system, the non-transitory computer-readable storage medium having instructions stored thereupon which, when executed by a processor, cause the processor to:
obtain a medicine delivery schedule associated with a patient;
identify a medicine and a medicine delivery time based on the medicine delivery schedule;
output a notification at the medicine delivery time to indicate to the patient to collect the medicine;
determine that the patient is in proximity to a dispensing system front portion responsive to outputting the notification; and
cause a compartment of a plurality of compartments to move from a closed position to an open position to enable the patient to take the medicine responsive to determining that the patient is in proximity to the dispensing system front portion, wherein the plurality of compartments is configured to store medicines associated with the patient.
